SEQUENCE LISTING (6 sequences)

SEQ ID NO: 1 - protein, 4 aa, synthetic construct
FAHV

SEQ ID NO: 2 - RNA, 12 nt, synthetic construct
  source 1..12: mol_type = other RNA
tttgcacatgtt

SEQ ID NO: 3 - RNA, 12 nt, synthetic construct
  source 1..12: mol_type = other RNA
ttcgcgcacgtt

SEQ ID NO: 4 - protein, 4 aa, synthetic construct
KGAI

SEQ ID NO: 5 - RNA, 12 nt, synthetic construct
  source 1..12: mol_type = other RNA
aggggcgcgatc

SEQ ID NO: 6 - RNA, 12 nt, synthetic construct
  source 1..12: mol_type = other RNA
cgcggcgcgatc

(19) United States
(12) Patent Application Publication
(10) Pub. No.: US 2026/0209794 A1
(43) Pub. Date: Jul. 23, 2026

(54) Compositions and Methods for Self-Adjuvanting AAV Vaccines

(71) Applicants: St. Jude Children's Research Hospital, Inc., Memphis, TN (US); University of Georgia Research Foundation, Inc, Athens, GA (US)

(72) Inventors: Stephen WINSTON, Memphis, TN (US); Andrew Davidoff, Memphis, TN (US); Kristin Wiggins, Memphis, TN (US); Stacey Schultz-Cherry, Memphis, TN (US); Ted M. Ross, Athens, GA (US)

(21) Appl. No.: 19/140,397

(22) PCT Filed: Dec. 21, 2023
(86) PCT No.: PCT/US2023/085309

§ 371 Date: Jun. 18, 2025

Related U.S. Application Data
(60) Provisional application No. 63/435,053, filed on Dec. 23, 2022.

(51) Int. Cl. C12N 15/86; A61K 39/00; A61K 39/145; A61P 37/04; C07K 14/005

(52) U.S. Cl. CPC C12N 15/86; A61K 39/145; A61P 37/04; C07K 14/005; A61K 2039/5256; A61K 2039/543; A61K 2039/575; C12N 2750/14143; C12N 2760/16122; C12N 2760/16134; C12N 2830/008

(57) ABSTRACT

A self-adjuvating recombinant, non-replicating Adeno-Associated Virus (AAV) vector harboring nucleic acids encoding one or more immunogenic peptides or proteins, wherein said nucleic acids include a plurality of immunostimulatory CpG motifs is provided. Also disclosed are immunogenic compositions and methods of eliciting an immune response using the AAV vector.

### INTRODUCTION

This application claims benefit from U.S. Provisional Patent Application Ser. No. 63/435,053, filed Dec. 23, 2022, the content of which is incorporated herein by reference in its entirety.

This invention was made with government support under 75N93019C00052 awarded by the National Institutes of Health. The government has certain rights in this invention.

### STATEMENT REGARDING ELECTRONIC FILING OF A SEQUENCE LISTING

A Sequence Listing in XML text format, submitted under 37 C.F.R. § 1.821-1.834, entitled “SJ0107WO” 6, 168 bytes in size, generated Dec. 8, 2023, is provided in lieu of a paper copy. This Sequence Listing is hereby incorporated by reference into the specification.

### BACKGROUND

Immune responses are highly desirable when they protect the body from infection by a microorganism or control the growth of tumors and other hyperproliferative disorders. To generate an immune response, three components are needed: an antigen, an adjuvant, and a delivery method. The most common means for generating an immune response is by administering a vaccine. In the case of the tetanus vaccine, the antigen is heat-inactivated tetanus toxoid protein, the adjuvant is alum (i.e., hydrated aluminum potassium sulfate), and the delivery method is the needle and syringe used for the subcutaneous or intramuscular injection.

For other kinds of vaccines, notably viral vaccines such as measles, mumps, rubella, polio, and varicella vaccines, a live, replicating virus is used. The live virus is typically attenuated or weakened as part of its selection and production, but the live virus vaccine contains both the viral antigens (proteins, carbohydrates, or lipids from the virus) along with the means of delivery. In this case, the delivery component is intrinsic to the ability of the virus to enter cells, partially or completely replicate, and thereby lead to the generation of its antigens in the host. The live virus vaccine can also carry its own adjuvant and thereby elicit a strong immune response. A good example of this is the Yellow Fever vaccine 17D in which the live virus vaccine is capable of interacting with Toll-Like Receptors (TLRs) on dendritic cells and other antigen-presenting cells, thereby activating these cells to initiate and amplify an immune response.

On the other hand, some vaccines fail to provide sufficient adjuvant activity. A good example is the live replicating viral vaccine for Respiratory Syncytial Virus (RSV). About half of all children are infected by RSV during their first year of life. However, a formalin-fixed RSV virus vaccine failed to protect children. This was subsequently traced to inability of the vaccine to provide the appropriate adjuvant activity, specifically the activation of TLRs on antigen-presenting cells. Conversely, if TLR agonists were added to the ineffective RSV vaccine, it became sufficient to elicit strong protective immune responses.

As the example of the RSV vaccine demonstrates, there is a need in the field to provide an adjuvant when the vaccine alone is insufficient for stimulating antigen-presenting cells. Sometimes this can be provided by mixing an adjuvant with the vaccine, such as mixing the MF59 adjuvant with the vaccine for influenza. However, this approach is not suitable for all situations.

In many cases, there is a need for a method to provide the antigen and the adjuvant co-extensive in space and time. To do this, it is preferable to incorporate the adjuvant into the vaccine formulation. For example, the Yellow Fever 17D vaccine incorporates the viral antigens, the TLR agonist adjuvants, and the delivery method (cell entry mediated by viral proteins) into a single entity. As such, the antigen and adjuvant are provided co-extensive in space and time.

### SUMMARY OF THE INVENTION

This invention provides a recombinant, non-replicating Adeno-Associated Virus (AAV) vector having a promoter operably linked to nucleic acids encoding one or more immunogenic peptides or proteins (e.g., designed with the computationally optimized broadly reactive (COBRAs) methodology), wherein said nucleic acids include a plurality of immunostimulatory CpG motifs therein. In some aspects, the plurality of immunostimulatory CpG motifs are present in the promoter or coding region of the immunogenic peptides or proteins. In other aspects, the AAV is a single-stranded AAV (ssAAV) vector or a self-complementary AAV vector (scAAV). In further aspects, expression of the nucleic acids encoding the immunogenic peptides or proteins is under control of a tissue-specific promoter, e.g., a lung-specific promoter or muscle-specific promoter. In particular aspects, the immunogenic peptides or proteins are monovalent or multivalent Influenza virus antigens, e.g., a hemagglutinin, neuraminidase, nucleoprotein, matrix antigen, or combination thereof. In still further aspects, the nucleic acids encoding the immunogenic peptides or proteins are located downstream of promoter P5 and/or upstream of promoter P5 or 5′ inverted terminal repeat sequences of the AAV vector.

This invention also provides an immunogenic composition including the recombinant, non-replicating AAV vector in admixture with an acceptable carrier, wherein said immunogenic composition does not include an exogenous adjuvant. In some aspects, the immunogenic composition is formulated for intramuscular, intradermal, or intranasal administration. A method of eliciting an immune response in a subject is also provided by administering the immunogenic composition to the subject to elicit, e.g., a humoral response such as neutralizing antibodies.

### BRIEF DESCRIPTION OF THE DRAWINGS

FIG. 1 depicts the ssAAV2/8 and scAAV2/8 vectors produced with nucleic acids encoding for COBRA HA, as well as the dosing and timing of administration and challenge with influenza virus.

FIG. 2 shows the HAI (hemagglutination inhibition) antibody titers pre- and post-challenge with lethal influenza virus in BALB/C mice primed with SSAAV2/8-COBRA-HA and SCAAV2/8-COBRA-HA vaccines (without adjuvant).

FIG. 3 shows the survival of BALB/C mice primed with SSAAV2/8-COBRA-HA and scAAV2/8-COBRA-HA vaccines (without adjuvant) and challenged six weeks later with lethal influenza virus.

FIGS. 4A-4E show that ssAAV2/9 vector-mediated delivery of wild-type and COBRA HA antigens provides better protection against morbidity and mortality as compared to FLUCELVAX® seasonal influenza vaccine. FIG. 4A depicts the ssAAV2/9 vectors produced with nucleic acids encoding for wild-type and COBRA HA. FIG. 4B shows the dosing and timing of administration and challenge with influenza virus. FIG. 4C shows a stronger vaccine-induced antibody response than the FLUCELVAX® seasonal influenza vaccine. FIG. 4D shows superior survival in AAV groups compared to controls. FIG. 4E shows superior pre-challenge HAI titers compared to controls.

FIGS. 5A-5D show the longevity of protection afforded by the AAV vector-mediated delivery of COBRA HA antigen. FIG. 5A shows the dosing and timing of administration and challenge with influenza virus. FIG. 5B shows survival in the AAV groups compared to controls. FIGS. 5C-5D show that antibody titers were maintained over time and provided protection against viral challenge. PV, post-vaccination.

FIGS. 6A-6C show that AAV2/9 vector-mediated delivery of COBRA HA antigen provides better protection against mortality as compared to FLUCELVAX® seasonal influenza vaccine in female mice. FIG. 6A shows the dosing and timing of administration and challenge with influenza virus. FIG. 6B shows superior pre- and post-challenge HAI titers compared to controls. FIG. 6C shows superior survival in AAV groups compared to controls.

FIG. 7 shows the HAI antibody titers pre-challenge (6 weeks post-vaccination) and post-challenge (9 weeks post-vaccination) with lethal influenza virus in mice primed with vaccines containing different capsids. N=10.

FIG. 8 shows the HAI antibody titers pre- and post-challenge with lethal influenza virus in mice primed with CpG-Low (V3: ssAAV2/9-COBRA HA vector, 162 total CpG), CpG-Medium V4: ssAAV2/9-COBRA HA vector, 242 total CpG), and CpG-High (V5: ssAAV2/9-COBRA HA vector, 345 total CpG) vectors. Mice were vaccinated at a dose of 5e8.

FIGS. 9A-9B show that AAV2/9 vector-mediated delivery of COBRA HA antigen intranasally provides protection against challenge with a lethal dose of influenza virus. FIG. 9A shows pre- and post-challenge HAI titers compared to controls. FIG. 9B shows survival in AAV groups.

FIG. 10 shows HAI antibody titers, which indicate that AAV2/9 vector-mediated delivery of COBRA HA antigen gives a better breadth of antibody responses across other H1N1 strains compared to antibody responses against wild-type HA at nine weeks post-vaccination.

FIG. 11 shows HAI antibody titers, which indicate that a robust breadth of antibody responses is generated regardless of the capsid. All vectors expressed COBRA HA (CpG-Low). Measurements were taken at six weeks post-vaccination.

FIGS. 12A-12C show that AAV2/9 vector-mediated delivery of multiple antigens provides protection against challenge with a lethal dose of influenza virus. FIG. 12A shows NAI titers in mice vaccinated with a vector expressing N1 or a combination of N1+H1. FIG. 12B shows survival in AAV groups. FIG. 12C shows NAI titers in mice vaccinated with a vector expressing N2 or a combination of N2+H3.

### DETAILED DESCRIPTION OF THE INVENTION

To enhance immune responses to one or more antigens or immunogenic proteins or peptides, this invention provides a self-adjuvating recombinant vector having a promoter operably linked to heterologous nucleic acids encoding the antigens or immunogenic peptides or proteins, wherein the heterologous nucleic acids and optionally the vector have been modified to include a plurality of immunostimulatory CpG motifs therein. In particular, the invention provides a recombinant non-replicating Adeno-Associated Virus (AAV) as a delivery vector for heterologous nucleic acids enriched in immunostimulatory CpG motifs, which encode one or more immunogenic peptides or proteins.

As used herein, “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), avian AAV, bovine AAV, canine AAV, equine AAV, primate AAV, non-primate AAV, and ovine AAV. “Primate AAV” refers to AAV capable of infecting primates, “non-primate AAV” refers to AAV capable of infecting non-primate mammals, “bovine AAV” refers to AAV capable of infecting bovine mammals, etc.

The abbreviation “rAAV” refers to recombinant adeno-associated virus, also referred to as a recombinant AAV vector (or “rAAV vector”). A “recombinant AAV vector” or “AAV vector” refers to a polynucleotide vector including one or more heterologous sequences (i.e., nucleic acids not of AAV origin) that are flanked by one or two 145 bp inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a rAAV vector is incorporated into a larger polynucleotide (e.g., in another vector such as a plasmid used for cloning or transfection), then the rAAV vector may be referred to as a “pro-vector” can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector genome can be packaged into an AAV virus capsid to generate a recombinant adeno-associated viral particle (rAAV particle).

In one aspect, the AAV of the invention is a single-stranded AAV (e.g., ssAAV). In addition to ssAAV, the invention also provides self-complementary AAV (scAAV). The scAAV vector genome includes DNA strands that anneal together to form double-stranded DNA. By skipping second strand synthesis, scAAV can be rapidly expressed in cells. In another aspect, the AAV of the invention is a scAAV. In accordance with this aspect, the vector includes a first nucleic acid harboring the heterologous nucleic acids and a second nucleic acid harboring a complement of the first nucleic acid, wherein the first nucleic acid can form intrastrand base pairs with the second nucleic acid along most or all its length. In some aspects, the first nucleic acid and the second nucleic acid are linked by a mutated AAV ITR, wherein the mutated AAV ITR includes a deletion of the D region and a mutation of the terminal resolution sequence.

Preferably, the AAV vector of the invention is non-replicating. In this respect, the AAV vector is replication-defective and lacks in its viral genome sequences encoding functional Rep (replication) and Cap (encapsidation) proteins. The defective AAV vector may lack most or all of the rep and cap coding sequences (which may be provided in trans) and carry essentially only one or two ITR sequences, which flank at least one control sequence operably linked to at least one heterologous nucleic acid.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters, enhancers and degrons. Non-limiting examples of control elements include sequences for transcription initiation and/or termination, promoter and/or enhancer sequences, efficient RNA processing signals (e.g., splicing and polyadenylation signals), sequences that stabilize cytoplasmic mRNA, sequences that enhance translation efficiency (e.g., Kozak consensus sequences), sequences that enhance protein stability, and/or sequences that enhance protein processing and/or secretion. Not all control elements need always be present, so long as the coding sequence of the heterologous nucleic acid(s) is capable of replication, transcription and/or translation in an appropriate host cell.

In certain aspects, the control element of the AAV vector is a promoter. A promoter is a DNA region capable of binding RNA polymerase and initiating transcription of a coding sequence. In this respect, a promoter is “operably linked” or “operatively linked” to a nucleic acid when it is in the correct location and orientation in relation to the heterologous nucleic acids to control RNA polymerase initiation and expression of the heterologous nucleic acid.

AAV contains four known promoters within its genome: P5, P19, P40 and P81. The P5 promoter is approximately 145 bp in length and located at the 5′ of the AAV genome, slightly downstream of the 5′ ITR sequence. The P5 promoter is present in all catalogued serotypes of AAV, although in AAV5, it is referred to as P7. According to some aspects, the promoter operatively linked to the heterologous nucleic acids (i.e., immunogenic protein or peptide) is the endogenous AAV P5 promoter or a functional portion thereof (e.g., a 135 bp portion). To increase the transcription level of the heterologous nucleic acids, the P5 promoter in the recombinant AAV vector can be replaced with, e.g., the cytomegalovirus (CMV) promoter, β-actin promoter, or a simian virus 40 (SV40) promoter. Alternatively, the heterologous nucleic acids may be operatively linked to an RSV LTR, a MoMLV LTR, a phosphoglycerate kinase-1 (PGK) promoter, a CK6 promoter, a transthyretin promoter (TTR), a TK promoter, a tetracycline responsive promoter (TRE), an HBV promoter, an hAAT promoter, a LSP promoter, a chimeric liver-specific promoter (LSP), an E2F promoter, a telomerase (hTERT) promoter, a cytomegalovirus enhancer/chicken beta-actin/Rabbit β-globin promoter (CAG) promoter, an elongation factor 1-alpha promoter (EF1-alpha) promoter, a human β-glucuronidase promoter, a chicken β-actin (CBA) promoter, a retroviral Rous sarcoma (RSV) virus LTR promoter, a dihydrofolate reductase promoter, and a β-actin promoter.

In some aspects, the heterologous nucleic acids are operably linked to a tissue-specific promoter, which limits the expression of the immunogenic protein or peptide to one or more cells or tissues. In some aspects, the tissue is lung tissue or muscle tissue. Accordingly, in certain aspects, the heterologous nucleic acids are operably linked to a lung-specific promoter or muscle-specific promoter. Lung-specific promoters include, but are not limited to, the surfactant protein C promoter, surfactant protein A1 promoter or the surfactant protein B promoter. Examples of muscle-specific promoters include, but are not limited to, alpha-actin, cardiac troponin C, myosin light chain 2A, skeletal beta-actin, CK6, dystrophin, muscular creatine kinase, dMCK, tMCK, enh348MCK, synthetic C5-12 (Syn), Myf5, MLC1/3f, MyoD1, Myog, or Pax7 promoters, as well as synthetic muscle promoters with activities higher than naturally occurring promoters (see, Li et al., (1999) Nat. Biotech. 17:241-245).

The nucleic acids inserted into the AAV vector are heterologous to the AAV vector. As used herein, “heterologous” means derived from a genotypically distinct entity from the rest of the entity to it is being compared too. For example, a nucleic acid introduced by genetic engineering techniques into a vector derived from a different species is a heterologous nucleic acid. In particular, an AAV including heterologous nucleic acids encoding a heterologous gene product is an AAV including nucleic acids not normally included in a naturally occurring, wild-type AAV, and the encoded heterologous nucleic acid products are products not normally encoded by a naturally occurring, wild-type AAV. Once transferred/delivered into a host cell, heterologous nucleic acids, contained within the AAV vector, can be expressed (e.g., transcribed, and translated if appropriate).

In accordance with the present invention, the AAV vector harbors one or more heterologous nucleic acids encoding immunogenic proteins or peptides. The term “polypeptide,” “peptide,” or “protein” refers to a polymer of amino acids. An “immunogenic protein,” “immunogenic peptide,” “immunogen,” or “antigen” refers to a molecule that is capable of inducing an adaptive immune response in an individual, where said adaptive immune response targets the immunogenic protein, immunogenic peptide, immunogen, or antigen. In relation to the present invention, an immunogenic protein, immunogenic peptide, immunogen, or antigen will induce a humoral and/or cell-mediated immune response. Immunogenic proteins or peptides are useful in generating an immune response (i.e., useful as, for example, a vaccine) in a human or non-human animal against pathogens including archaea, bacteria, viruses, protozoans, mycoplasma, fungi, parasitic microorganisms, or multicellular parasites infecting human and non-human vertebrates, or from a cancer cell or tumor cell. Immunogenic proteins or peptides of the invention can be obtained from a single source (i.e., a monovalent antigen) or can be used in combination as individually expressed proteins or peptides (e.g., via Internal ribosome entry sites (IRESs)), or as a fusion protein (i.e., a multivalent antigen). In some aspects, a fusion protein can be cleaved to provide individual immunogenic proteins or peptides using, e.g., a self-cleaving peptide such as porcine teschovirus-1 2A (P2A), Thosea asigna virus 2A (T2A), equine rhinitis A virus 2A (E2A), or foot-and-mouth disease virus 2A (F2A).

Immunogenic proteins or peptides can be derived from bacterial pathogens including, e.g., Legionella pneumophila, Listeria monocytogenes, Campylobacter jejuni, Staphylococcus aureus, Streptococcus pneumoniae, Streptococcus pyogenes, Borrelia burgdorferi, Helicobacter pylori, Ehrlichia chaffeensis, Clostridium difficile, Vibrio cholerae, Salmonella enterica, Salmonella typhi, Bartonella henselae, Chlamydia pneumoniae, Clostridium botulinum, Clostridium perfringens, Vibrio vulnificus, Parachlamydia, Corynebacterium amycolatum, Corynebacterium diphtheria, Klebsiella pneumoniae, Klebsiella granulomatis, Acinetobacter baumannii, Pseudomonas aeruginosa, Neisseria gonorrhoeae, Neisseria meningitidis, Bordetella pertussis, Haemophilus influenzae, Shigella dysenteriae, Bacillus anthracis, Mycobacterium ulcerans, Mycobacterium leprae, Mycobacterium tuberculosis, Mycobacterium paratuberculosis, Capnocytophaga canimorsus, Elizabethkingia sp., Burkholderia mallei, Burkholderia pseudomallei, Erysipelothrix rhusiopathiae, Francisella tularensis, Rickettsia conorii, Rickettsia, Leptospira sp., Nocardia sp., Yersinia pestis, and Streptobacillus moniliformis. In some aspects, the immunogenic protein is a bacterial surface protein, or immunogenic peptide thereof. Examples of antigens from bacterial pathogens include, e.g., tuberculosis antigens such as Phosphate transport receptor PstS-3 (Ag88), Catalase-peroxidase (KatG), and Antigen MPT63; Neisseria surface protein A (NspA) and Transferrin binding protein (TbpA) obtained from N. meningitidis; Protective Antigen (PA) derived from B. anthracis; Pertussis toxin S1 sub-unit, Filamentous haemagglutinin and Pertactin (P69) from B. pertussis; Outer surface protein A, B or C from B. burgdorferi; Flagellin (FlaA) from C. jejuni; Major outer membrane protein (MOMP) from C. trachomatis; Phospholipase D from C. pseudotuberculosis; Fimbrin (P5), Outer membrane protein P1 or P6 from H. influenzae; Heat shock protein 10, Neutrophil-activating protein A, Citrate synthase, Urease or Catalase from H. pylori; Major secretory protein, Heat shock protein 60 or Outer membrane protein S from L. pneumophila; Listeriolysin-O or Major extracellular protein (P60) from L. monocytogenes; Outer membrane protein F (OprF) from Pseudomonas aeruginosa; Penicillin-binding protein (MecA), Fibrinogen binding protein, and Collagen adhesin from S. aureus; Pneumococcal surface protein A (PspA) and Pneumolysin from S. pneumoniae; Fibronectin binding protein from S. pyogenes; and V antigen from Y. pestis.

Immunogenic proteins or peptides can be derived from viral pathogens including, e.g., human immunodeficiency virus (HIV; e.g., HIV-1 and HIV-2), influenza (e.g., influenza A, influenza B, and influenza C), parainfluenza hepatitis virus (e.g., hepatitis A, hepatitis B, hepatitis C, hepatitis D, and hepatitis E), herpes viruses (e.g., HSV-1; HSV-2), varicella-zoster virus (HHV-3), Epstein Barr virus (HHV-4), Roseolovirus (HHV-6A and HHV-6B); Rous sarcoma virus, cytomegalovirus (HHV-5), Kaposi's sarcoma-associated herpesvirus, human papilloma virus (e.g., HPV-1, HPV-2, HPV-16, and HPV-18), parvovirus (e.g., Parvovirus B19), orthomyxovirus, paramyxovirus (e.g., morbillivirus, respirovirus, rubulavirus, ferlavirus, pneumovirus, and metapneumovirus), picornaviruses (e.g., foot-and-mouth disease virus, aquamavirus, encephalomyocarditis virus, theilovirus, cosavirus, cadicivirus, enterovirus, rhinovirus aichivirus, melegrivirus, human parechovirus, and salivirus), togaviruses (e.g., flavivirus, alphavirus, and rubivirus), Cowpox virus, Horsepox virus, Crimean-Congo hemorrhagic fever virus, Dengue virus, Eastern equine encephalitis virus, Ebola virus, Hantaan virus, Human coronavirus, Human enterovirus 68, Human enterovirus 70, non-HIV retroviruses, rhinovirus, respiratory syncytial virus (RSV), SARS coronavirus, Human T-lymphotropic virus, Japanese encephalitis virus, Lassa virus, Lymphocytic choriomeningitis virus, MERS coronavirus, measles virus, Mengo encephalomyocarditis virus, Monkeypox virus, mumps virus, Norwalk virus, Pichinde virus, Poliovirus, Rabies virus, rotavirus, Rubella virus, St. louis encephalitis virus, Toscana virus, Uukuniemi virus, Venezuelan equine encephalitis virus, Western equine encephalitis virus, West Nile virus, Yellow fever virus, and ZIKA virus, as well as any other viruses known to those of skill in the art. Examples of viral antigens include, e.g., HIV antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens as such hemagglutinin, neuraminidase, nucleoprotein, matrix and other influenza viral components; SARS coronavirus spike protein; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpl, gpll, and other varicella zoster viral antigen components; Japanese encephalitis viral antigens such as proteins E, M-E, M-E-NSI, NSI, NS1-NS2A, 80% E, and other Japanese encephalitis viral antigen components; rabies viral antigens such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens. In particular aspects, the immunogenic peptide or protein is an influenza virus antigen. In certain aspects, the immunogenic peptide or protein is one or more of hemagglutinin (HA), neuraminidase (NA), nucleoprotein, or matrix antigen derived from an influenza virus. In particular aspects, the influenza antigen is monovalent. In other aspects, the influenza antigen is multivalent, e.g., a combination of HA and NA antigens.

Immunogenic proteins or peptides can be derived from pathogenic protozoans and helminths including, e.g., Entamoeba histolytica, Plasmodium sp. (e.g., P. falciparum), Leishmania sp. Toxoplasma gondii, Pneumocystis carinii, Trypanosoma sp., Babesia sp., Giardia lamblia, Filarioidea sp., Schistosoma sp., Platyhelminthes sp., or Cestoidea sp. Examples of antigens from pathogenic protozoans and helminths include, e.g., PfMSA180 or Circumsporozoite protein (CSP) from P. falciparum, 5G8 protein from G. lamblia, rhoptry proteins 2 and 4 (ROP2 and ROP4), surface antigen 1 (SAG1) and AMA from T. gondii.

Immunogenic proteins or peptides can be derived from fungal pathogens including, e.g., Candida sp. (e.g., C. albicans), Malassezia sp., Aspergillus sp., Cryptococcus sp. (e.g., C. neoformans), Histoplasma sp. (e.g., H. capsulatum) and Zygomycetes sp. Examples of antigens from pathogenic fungi include, e.g., Als3p, Als1p, SAP2, fructose biphosphate aldolase, and cell surface protein Hyr1 from C. albicans; Histone H2B-like protein, heat shock protein 60, HIS-62, 80-kilodalton antigen, Sec31 antigen, or H antigen from H. capsulatum; or mannoprotein from C. neoformans.

Examples of cancer antigens that may be used by the present invention include, e.g., IGF-IR, CanAg, EGF-R, EGF-RvIII, EphA2, MUC1, MUC16, VEGF, TF, CD19, CD20, CD22, CD27, CD33, CD37, CD38, CD40, CD44, CD56, CD70, CD138, CA6, Her2/neu, CRIPTO (a protein produced at elevated levels in a majority of human breast cancer cells), alphaᵥ/beta₃ integrin, alphaᵥ/beta₅ integrin, TGF-β, CDlla, CD18, Apo2, EpCAM and C242. In some aspects, the antigen is a cellular oncogene, such as ras or myc.

In some aspects of this invention, the immunogenic protein or peptide is a computationally optimized broadly reactive antigen (COBRA). This methodology provides a more broadly reactive antigen as it uses multiple rounds of layered consensus building to generate an antigen from multiples strains or subtypes, e.g., influenza HA immunogens for H1, H3, and H5 influenza subtypes. By way of illustration, COBRA HA antigens are capable of eliciting broadly reactive HA-specific antibody responses that can protect against both seasonal and pandemic influenza strains that have undergone genetic drift.

An antigen or immunogenic protein or peptide is derived from its source when it is isolated or recombinantly produced or expressed in a recombinant expression system. A skilled artisan realizes that any DNA, which contains nucleotide sequences or partial nucleotide sequences of a pathogenic genome or a gene or a fragment of a gene for a protein that elicits an immune response, results in synthesis of an antigen. Furthermore, one skilled in the art realizes that the present invention is not limited to the use of the entire nucleic acid sequence of a gene or genome. In this respect, partial nucleic acid sequences of more than one gene or genome may be used, and these nucleic acid sequences may be arranged in various combinations to elicit the desired immune response.

In accordance with this invention, the recombinant AAV vector is self-adjuvating. In this respect, the recombinant AAV vector can elicit an immune response to an immunogenic protein or peptide in a subject in the absence of an exogenous adjuvant. The self-adjuvating recombinant AAV vector is prepared by incorporating a plurality of CpG motifs in the nucleic acid sequences encoding the one or more immunogenic proteins or peptides and/or the promoter sequence operably linked to said nucleic acids. Optionally, other sequences in the AAV vector As is known in the art, a “CpG motif” is a cytosine triphosphate deoxynucleotide (“C”) followed by a guanine triphosphate deoxynucleotide (“G”). The “p” refers to the phosphodiester link between consecutive nucleotides. When these CpG motifs are unmethylated, they act as immunostimulants. CpG motifs are considered pathogen-associated molecular patterns (PAMPs) due to their abundance in microbial genomes but their rarity in vertebrate genomes. The CpG PAMP is recognized by the pattern recognition receptor Toll-Like Receptor 9 (TLR9), which is constitutively expressed in B cells and plasmacytoid dendritic cells (pDCs) in humans and other higher primates.

CpG motifs can be incorporated into nucleic acids, in particular coding sequences, by modifying the codon used for each individual amino acid residue as well as adjacent codons. For example, for amino acid residues such as Ser, Pro, Thr, Ala and Arg, codons including CG are used, i.e., UCG, CCG, ACG, GCG and CGA, respectively. Further, codons of two adjacent amino acid residues can be modified to increase the number of CGs. For example, the sequence Phe-Ala-His-Val (SEQ ID NO: 1) encoded by the sequence UUU·GCA·CAU·GUU (SEQ ID NO: 2) can be modified to UUC·GCG·CAC·GUU (SEQ ID NO: 3) so that the sequence includes the three CpG G motifs. Using the conventional codon table set forth below in Table 1, the skilled artisan can readily convert a nucleic acid sequence with very few CpG motifs to a nucleic acid sequence with a plurality of CpG motifs.

TABLE 1

| | | | | | | | |
|---|---|---|---|---|---|---|---|
| UUU | Phe | UCU | Ser | UAU | Tyr | UGU | Cys |
| UUC | | UCC | | UAC | | UGC | |
| UUA | Leu | UCA | | UAA | Stop | UGA | Stop |
| UUG | | UCG | | UAG | | UGG | Trp |
| CUU | Leu | CCU | Pro | CAU | His | CGU | Arg |
| CUC | | CCC | | CAC | | CGC | |
| CUA | | CCA | | CAA | Gln | CGA | |
| CUG | | CCG | | CAG | | CGG | |
| AUU | Ile | ACU | Thr | AAU | Asn | AGU | Ser |
| AUC | | ACC | | AAC | | AGC | |
| AUA | | ACA | | AAA | Lys | AGA | Arg |
| AUG | Met | ACG | | AAG | | AGG | |
| GUU | Val | GCU | Ala | GAU | Asp | GGU | Gly |
| GUC | | GCC | | GAC | | GGC | |
| GUA | | GCA | | GAA | Glu | GGA | |
| GUG | | GCG | | GAG | | GGG | |

In addition, it is contemplated that a protein sequence, which is encoded by a nucleic acid sequence that is not amenable to the incorporation of a plurality of CpG motifs, could be modified by conservative amino acid substitutions to increase the number of possible CpG motifs. For example, whereas the amino acid sequence Lys-Gly-Ala-Ile (SEQ ID NO:4), encoded by, e.g., codons AGG·GGC·GCG·AUC (SEQ ID NO: 5), has only two CpG motifs possible, substitution of Lys with Arg provides a sequence with four CpG motifs, i.e., CGC·GGC·GCG·AUC (SEQ ID NO: 6).

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

In some aspects, a plurality of CpG motifs refers to a number of CpG motifs present in a nucleic acid sequence. In accordance with this aspect, a plurality of CpG motifs is at least 100, 120, 140, 160, 180, 200, 220, 240, 260, 280, 300, 320, 340, 360, 380, 400, 420, 440, 460, 480, or 500 CpG motifs. In another aspect, a plurality of CpG motifs refers to a percentage of possible nucleotides that could be changed to incorporate CpG motifs. For example, in the sequence UUU·GCA·CAU·GUU (SEQ ID NO:2), there are three possible modifications that can be made to incorporate CpG motifs without altering the encoded amino acid sequence, i.e., UUC·GCG·CAC·GUU (SEQ ID NO: 3). In some aspects, at least 50%, 55%, 60%, 65%, 70%, 75%, 80%, 85%, 90%, 95% or 100% of the possible nucleotides that could be changed to incorporate CpG motifs are changed. In accordance with either of the above aspects, the wild-type nucleic acid sequence encoding the immunogenic protein or peptide of interest is modified to include at least 20%, 25%, 30%, 35%, 40%, 45%, 50%, 55%, 60%, 65%, 70%, 75%, 80%, 85%, 90%, 95% or 100% more CpG motifs than what is present in the wild-type nucleic acid sequence thereby increasing the immune response to the immunogenic protein or peptide as compared to the immune response to the immunogenic protein or peptide encoded by the wild-type nucleic acid sequence.

Nucleic acids encoding the one or more immunogenic peptides or proteins can be inserted into the AAV vector at a position (i) downstream of promoter P5 of the AAV vector; and/or (ii) upstream of promoter P5 or 5′ inverted terminal repeat sequences of the AAV vector. In some aspects, the AAV vector can include nucleic acids encoding a first immunogenic protein inserted downstream (3′) of the P5 promoter of the AAV vector and also include nucleic acids encoding a second immunogenic protein (e.g., a different antigen from the same pathogen or a different pathogen) inserted upstream (5′) of the P5 promoter of the AAV vector. P5 is a bidirectional promoter (Stutika et al. (2016) J. Virol. 90:1278-1289) and insertion of reporter (GFP) sequences upstream of P5 have been shown to be packaged as contaminants in rAAV and expressed in vivo. (Brimble et al. (2022) Mol. Ther. Meth. Clin. Dev. 24: P280-291).

In some aspects, a nucleic acid insert or packaged nucleic acids is at least 50, 100, 200, 300, 400, 500, 600, 700, 800, 900, 1000, 1100, 1200, 1300, 1400, or 1500 nucleic acids in length. In certain aspects, a nucleic acid inserted downstream (3′) of the P5 promoter of the AAV vector is at least 50, 100, 200, 300, 400, 500, 600, 700, 800, 900, 1000, 1100, 1200, 1300, 1400, or 1500 nucleic acids in length. In other aspects, a nucleic acid inserted upstream (5′) of promoter P5 or 5′ inverted terminal repeat sequences of the AAV vector is at least 50, 100, 200, 300, 400, 500, 600, 700, 800, 900, 1000, 1100, 1200, 1300, 1400, or 1500 nucleic acids in length.

Methods of generating and/or modifying AAV to provide the recombinant AAV vectors herein are well-known in the art. See, e.g., WO 2000/28004; WO 2001/23001; WO 2004/112727; WO 2005/005610 and WO 2005/072364. Likewise, methods for packaging and assembly of AAV virions or AAV particles are also known in the art, and any conventional helper virus allowing AAV to be replicated and packaged by a host cell (e.g., a mammalian host cell such as murine cells and primate cells) can be used in accordance with this invention, including, e.g., adenoviruses, herpesviruses, and poxviruses such as vaccinia. Introduction of the AAV vector into the host cell may also be accomplished using techniques known to the skilled artisan. In a preferred aspect, standard transfection techniques are used, e.g., CaPO₄ transfection or electroporation, and/or infection by hybrid adenovirus/AAV vectors into cell lines such as the human embryonic kidney cell line HEK293 (a human kidney cell line containing functional adenovirus E1 genes providing trans-acting E1 proteins).

An “AAV virion” or “AAV virus” or “AAV viral particle” or “AAV vector particle” refers to a viral particle composed of at least one AAV capsid polypeptide and an encapsidated AAV vector including a heterologous nucleic acid (i.e., a nucleic acid other than a wild-type AAV genome, such as a transgene encoding an immunogenic protein or peptide). Thus, production of AAV virion or AAV particle necessarily includes production of AAV vector as such a vector is contained within an AAV virion or AAV particle.

The present disclosure also provides an immunogenic composition comprising the AAV vector in admixture with an acceptable carrier. In particular aspects, immunogenic composition does not include an exogenous adjuvant, e.g., Freunds adjuvant, Saponin, aluminium hydroxide, N-acetyl-nor-muramyl-L-alanyl-D-isoglutamine, or RIBI. Acceptable carriers, diluents, or excipients include any agent that can be administered without undue toxicity. Suitable carriers include, but are not limited to, liquids such as water, saline, glycerol, and ethanol. Salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may also be included. Further, stabilizing agents such as recombinant human albumin may be included to increase vector stability at moderate temperatures. A wide variety of carriers are known in the art and need not be discussed in detail herein. Carriers have been amply described in a variety of publications, including, for example, A. Gennaro, (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

In certain aspects, the immunogenic composition is formulated for intravenous, subcutaneous, intraperitoneal, mucosal (particularly nasal) routes. The administration of the immunogenic composition may be by parenteral injection, for example, a subcutaneous, intradermal, or intramuscular injection. Immunogenic compositions can be formulated to be compatible with a particular route of administration or delivery, as set forth herein or known to one of skill in the art. The route of administration may also be dependent upon the AAV subtype as well as the immune response to be achieved. For example, the tropism of AAV2, AAV5, AAV9 and AAVrh.10 is for the lung such that the mucosal route may advantageously be used. By comparison, AAV1, AAV2, AAV8, AAV9 and AAVrh.74 exhibit muscle tropism such that intramuscular injection may advantageously be used. Moreover, when the immune response to be raised is against a respiratory virus, e.g., influenza, it may be suitable to administer the virus, in particular one with a lung tropism, via the mucosal route. By comparison, when the immune response to be raised is against a cancer antigen, e.g., intratumoral injection may be used. In certain aspects, the immunogenic composition is formulated for intramuscular, intradermal, or intranasal administration, e.g., in the form of an injectable, either as a liquid solution or suspension.

Doses can vary and depend upon the AAV vector or immunogenic protein or peptide, as well as the disease type, onset, progression, severity, or frequency of administration. Moreover, the dose may be dependent upon the clinical endpoint desired, previous or simultaneous treatments, the general health, age, gender, race, or immunological competency of the subject and other factors that will be appreciated by the skilled artisan. The dose amount, number, frequency, or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing an immunological benefit. In some aspects, the inclusion of CpG motifs in the AAV vector or immunogenic protein or peptide allows for a reduced dose of the AAV vector or immunogenic protein or peptide. In some aspects, the inclusion of CpG motifs in the AAV vector or immunogenic protein or peptide allows for at least about a 10-fold to at least about a 1000-fold decrease (e.g., about a 10-, 20-, 30-, 40-, 50-, 60-, 70-, 80-, 90-, 100-, 200-, 300-, 400-, 500-, 600-, 700-, 800-, 900-, or 1000-fold decrease) in dose, as compared to an AAV vector or immunogenic protein or peptide that has not been optimized to include CpG motifs, without compromising protection against by an infection.

A therapeutic or beneficial effect of treatment is therefore any objective or subjective measurable or detectable improvement or benefit provided to a particular subject. A therapeutic or beneficial effect can, but need not be complete, ablation of all or any particular adverse symptom, disorder, illness, or complication of a disease. Thus, a satisfactory clinical endpoint is achieved when there is an incremental improvement or a partial reduction in an adverse symptom, disorder, illness, or complication caused by or associated with a disease, or an inhibition, decrease, reduction, suppression, prevention, limit or control of worsening or progression of one or more adverse symptoms, disorders, illnesses, or complications or caused by associated with the disease, over a short or long duration (hours, days, weeks, months, etc.).

The dose to achieve an effect, e.g., the dose in vector genomes/per kilogram of body weight (vg/kg), will vary based on several factors including, but not limited to, route of administration, the level of heterologous nucleic acid expression required to achieve an effect, the specific disease treated, a host immune response to the viral vector, a host immune response to the heterologous nucleic acid or expression product (protein or peptide), and the stability of the protein expressed. One skilled in the art can readily determine a dose range based on the aforementioned factors, as well as other factors. Generally, doses will range from at least about, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve an effect. In particular aspects, the dose may be reduced to 1×10⁸ vg/kg without compromising protection against infection by a pathogen, as compared to an AAV vector harboring nucleic acids encoding one or more immunogenic peptides or proteins, which does not include a plurality of immunostimulatory CpG motifs.

This invention also provides methods for using a recombinant AAV vector harboring nucleic acids encoding one or more immunogenic peptides or proteins, wherein said nucleic acids include a plurality of immunostimulatory CpG motifs, to stimulate the immune system to defend the host from pathogens or hyperproliferative diseases including cancer. More particularly, the present invention is a method for eliciting an immune response in a subject (e.g., an animal, in particular a mammal such as a human) by administering an immunogenic composition including the recombinant AAV vector to the subject.

The immune response may be a humoral and/or cell-mediated immune response (i.e., one mediated by T lymphocytes and/or other white blood cells, e.g., natural killer (NK) cells and macrophages). In certain aspects, the immune response is a humoral response to the immunogenic protein or peptide. In particular aspects, the humoral response is the production of neutralizing antibodies, which are capable of binding the immunogenic protein or peptide, and preferably preventing the initiation of infection and/or reducing the severity or intensity of an infection, e.g., by a pathogen such as an archaea, bacteria, virus, protozoan, mycoplasma, fungus, parasitic microorganism, or multicellular parasite. In some aspects, the immunogenic composition is administered in the form of a vaccine to stimulate a protective immune response against infection. Immunity to infection by a pathogen may be quantified using any appropriate technique, examples of which are known in the art.

In aspects, the recombinant AAV vector elicits an immune response that is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, preventing or inhibiting the progression of a cancer, or increasing the lifespan of a subject with a cancer. In some aspects, the cancer is a primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, bladder cancer, cervical cancer or the like.

The use of a self-adjuvating recombinant AAV enriched in immunostimulatory CpG motifs provides for an increase in the immune response to an immunogenic protein or peptide as compared to a recombinant vector which has not modified to include a plurality of CpG motifs. The terms “increased,” “increase,” or “enhanced” are all used herein to mean an increase by a statistically significant amount. These terms can mean an increase of at least 10% as compared to a reference level, for example, an increase of at least about 20%, 30%, 40%, 50%, 60%, 70%, 80%, 90% or up to and including a 100% increase or any increase between 10-100% as compared to a reference level, or at least about a 2-fold, 3-fold, 4-fold, 5-fold, 10-fold, or any increase between 2-fold and 10-fold or greater as compared to a reference level.

In addition to AAV, it is contemplated that the principles set forth herein can also be applied to other vectors and other antigens. For example, exogenous nucleic acids encoding one or more immunogenic proteins or peptides can be inserted into a herpes simplex virus vector, lentiviral vector, adenoviral vectors, plasmid, or as naked DNA and be modified to include an increased number of CpG motifs thereby enhancing the immune response to the immunogenic proteins or peptides encoded by the exogenous nucleic acids. Likewise, in view of the robust immune responses induced by the vectors disclosed herein, antigens from other respiratory viruses such as RSV, SARS-COV-2, and the like, can be incorporated into the vectors of this invention.

### Example 1: Influenza Vaccine Encoded by Nucleic Acids Enriched with CpG Motifs

A vaccine strategy was developed to use recombinant Adeno-Associated Viral Vectors (rAAV) to encode novel Computationally Optimized Broadly Reactive Antigens (COBRA) influenza antigens. The vaccine is administered intramuscularly or intranasally and results in the organism's own cells producing the antigen encoded by the vector to mount an immune response.

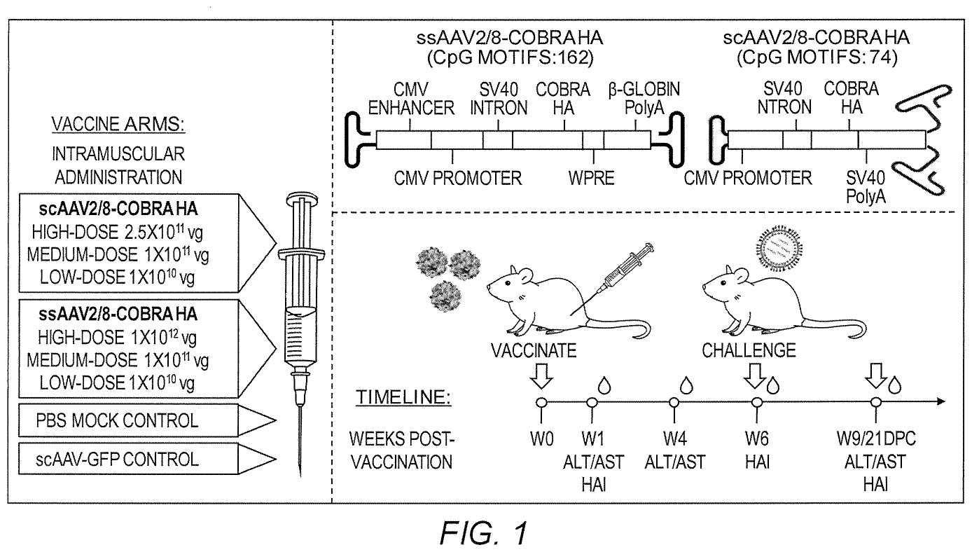

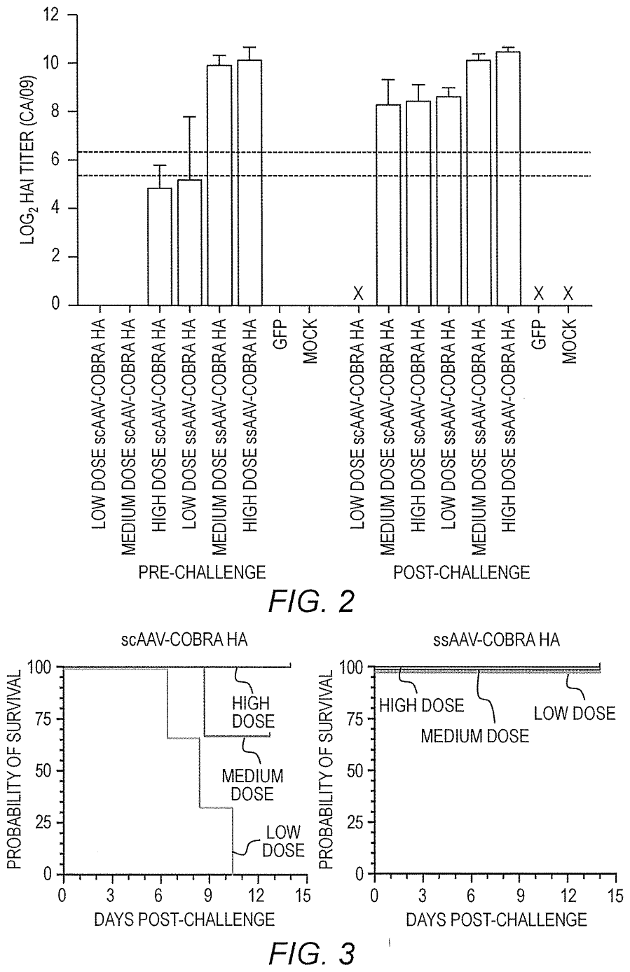

AAV2/8 vectors (ssAAV and scAAV) expressing COBRA HA influenza antigen were produced (FIG. 1) and the AAV2/8 capsid generated high pre-challenge antibody titers with a single dose (FIG. 2), without additional adjuvants. Mice vaccinated via intramuscular administration with these vectors exhibited complete survival upon challenge (FIG. 3), with minimal clinical symptoms. Similar experiments were conducted with intranasal administration with similar results. This is an advantage over the same recombinant protein, not included in an AAV vector, which requires three adjuvanted doses to achieve similar results (FIG. 9). In addition, the vector is superior to other rAAV influenza vaccines in that complete survival after virus challenge was achieved with a single dose at a lower vector concentration than, e.g., AAV9, which required three doses of 10¹¹ vg per animal intranasally to achieve complete survival (Demminger et al. (2020) EMBO Mol. Med. 12: e10938). This is the first example of COBRA antigens placed in an AAV vector and the first demonstration of using AAV2/8 in a vaccine context.

Additional rAAV vectors were produced to encode multiple antigens. Due to space constraints, conventional AAV vectors express a single antigen per vector. In contrast, vectors expressing COBRA H1+N1 and COBRA H1+H3 were prepared. In addition, the vector was modified to have an expression cassette composed of, in order: CMV promoter→synthetic 5′ UTR→SV40 intron→COBRA antigen→WPRE→BgH polyA.

To improve safety, the AAV vector is modified to replace conventional CMV, CBA or SV40 promoters with tissue-specific promoters thereby minimizing expression of the antigen in non-target tissue.

To increase the number of antigens that can be delivered, nucleic acids encoding one or more additional antigens are inserted outside of the expression cassette (i.e., outside or 5′ of P5) to be incorporated in the final product. In this respect, a single rAAV vector can express at least three antigens, e.g., HA/NA/NP. Moreover, a single rAAV vector can include combinations of antigens from different pathogens. For example, a combined Influenza, SARS-CoV2, and RSV rAAV vaccine can be produced.

To further enhance the immune response, the rAAV vector can also include nucleic acids encoding cytokines such as IL-15 and BAFF.

Advantages of using the rAAV vector include long-lasting immunity (e.g., greater than 88 weeks; Zabaleta et al. (2022) Mol. Ther. 30 (9): 2952-2967), ease of transportation at moderately cold temperatures (e.g., 4° C.), ease of altering the sequence of the insert to adjust for pathogen variants, and relatively fast production times as compared to egg-based vaccines.

Exemplary AAV2/9 expression constructs harboring nucleic acids encoding influenza COBRA HA antigen with enriched CpG motifs as compared to the unmodified nucleic acid sequences are presented in Table 2. The mid (50%) and high (100%) constructs generated refer to the percentage of possible locations for CpGs, without changing the amino acid sequence. Also, there are CpGs in the rest of the vector (promoter, intron, ITR) but the densities in these sites were not changed.

TABLE 2

| | | CpG Content in nucleic acids | |
|---|---|---|---|
| Vector | Construct | HA antigen | Total Vector |
| V3 | CpG Low | 26 | 162 |
| | (unmodified) | | |
| V4 | CpG Mid (50%) | 106 | 242 |
| V5 | CpG High (100%) | 209 | 345 |

### Example 2: Influenza Vaccine Provides Protection Against Mortality as Compared to Flucelvax

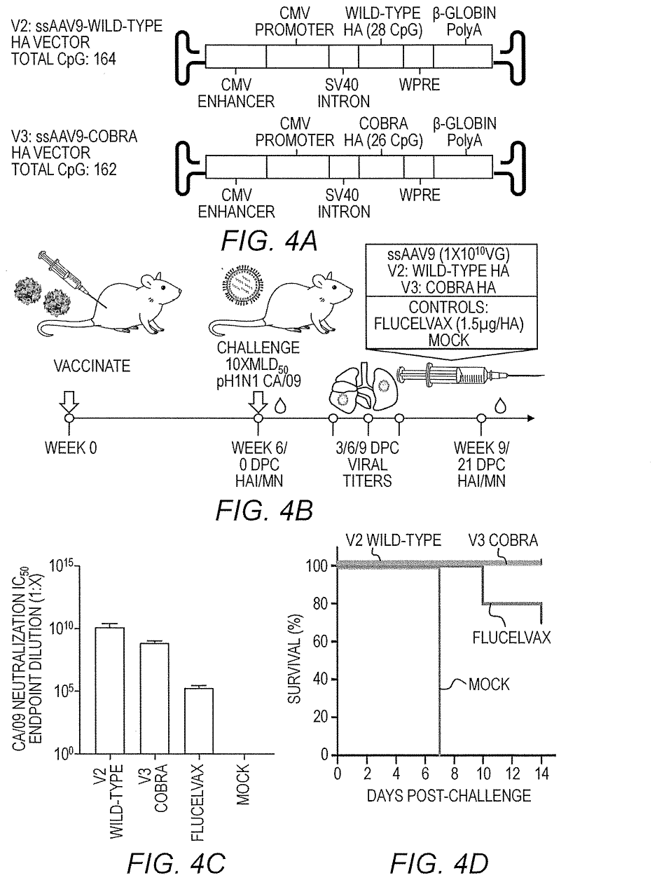

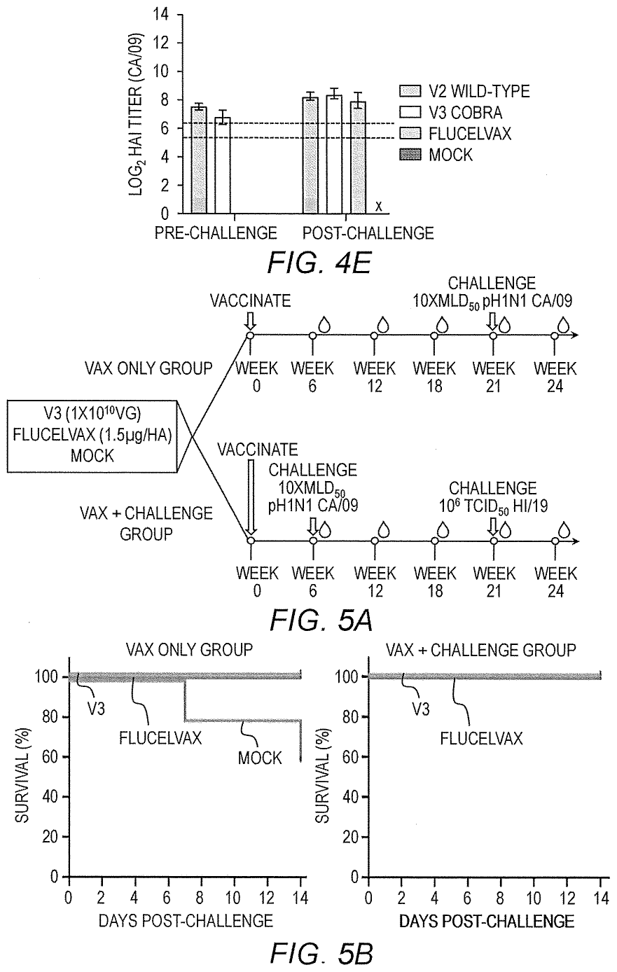

COBRA HA antigen was compared to a wild-type HA antigen (FIG. 4A). Mice were vaccinated with 1×10¹⁰ vg vector (FIG. 4B). Both vector's induced strongly neutralizing antibodies pre-challenge, and showed a stronger vaccine induced antibody response than the FLUCELVAX® seasonal influenza vaccine (FIG. 4C). In addition, both vectors offered better protection against morbidity; mice given AAV-based vaccines had no weight loss, while the FLUCELVAX® seasonal influenza vaccine and the mock vaccinated lost weight throughout the peak of the viral challenge. Further, the AAV groups exhibited superior survival as compared to controls (FIG. 4D). Notably, the HA AAV vaccines (COBRA HA antigen and wild-type HA antigen) controlled influenza replication in the nasal passages and lungs during challenge, while the FLUCELVAX® seasonal influenza vaccine and the mock controls did not. Moreover, pre-challenge HAI titers in the AAV vaccines were superior compared to the FLUCELVAX® seasonal influenza vaccine and the mock-vaccinated controls (FIG. 4E).

### Example 3: AAV Vaccine Exhibits Longevity

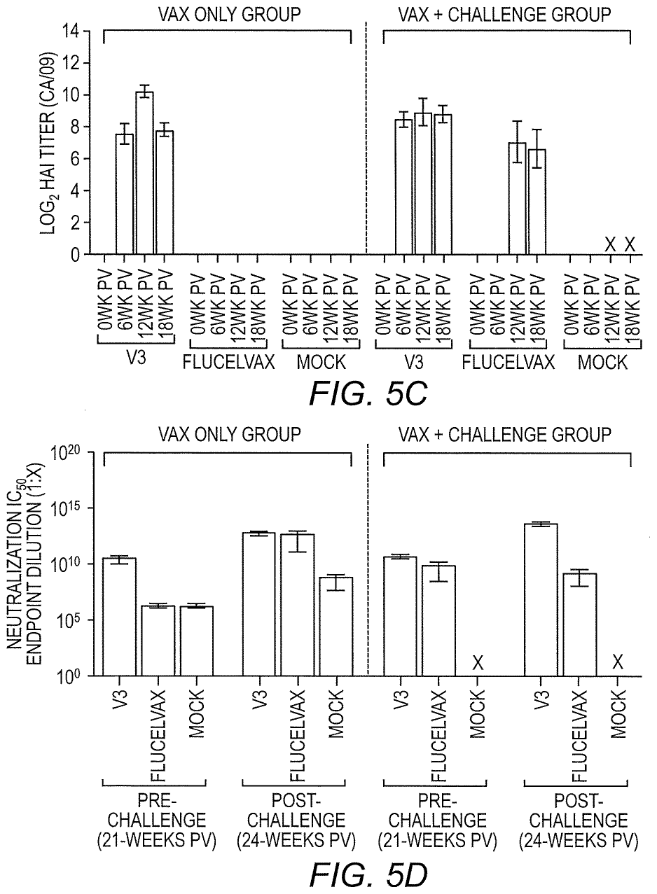

In this study, mice were vaccinated with 1×10¹⁰ vg of COBRA HA antigen vaccine (V3 vaccine, FIG. 4A). Half were challenged with 10XMLD₅₀ pH1N1 CA/09 (Vax+Challenge), while others were not (Vax Only) (FIG. 5A). Mice were held for 21 weeks to assess the longevity of protection. They maintained their antibody titers over that entire time, and complete protection from viral challenge against CA/09 was observed for the Vax Only group, or HI/19 (a drifted H1N1 strain) for the Vax+Challenge group (FIGS. 5B-5D).

In a similar study, mice were vaccinated with the CpG-enriched versions of the COBRA HA vector. In particular, mice were vaccinated with 1×10¹⁰ vg of CpG-Med (V4: ssAAV9-COBRA HA vector, 242 total CpG) and CpG-High (V5: ssAAV9-COBRA HA vector, 345 total CpG) vectors. Half were challenged with 10XMLD₅₀ pH1N1 CA/09 at week 6 post-vaccination, while the others were not. Mice were held for 21 weeks to assess the longevity of protection. The mice previously challenged at week 6 were challenged with 10⁶ TCID₅₀ HI/19, whereas the mice that were not previously challenged received 10⁶ XMLD₅₀ pH1N1 CA/09. All animals maintained their antibody titers over the entire time period, and complete protection (100% survival) from the viral challenge was observed in all mice. The outcomes were similar to the non-CpG enriched version.

### Example 4: Protection is Exhibited in Female Mice

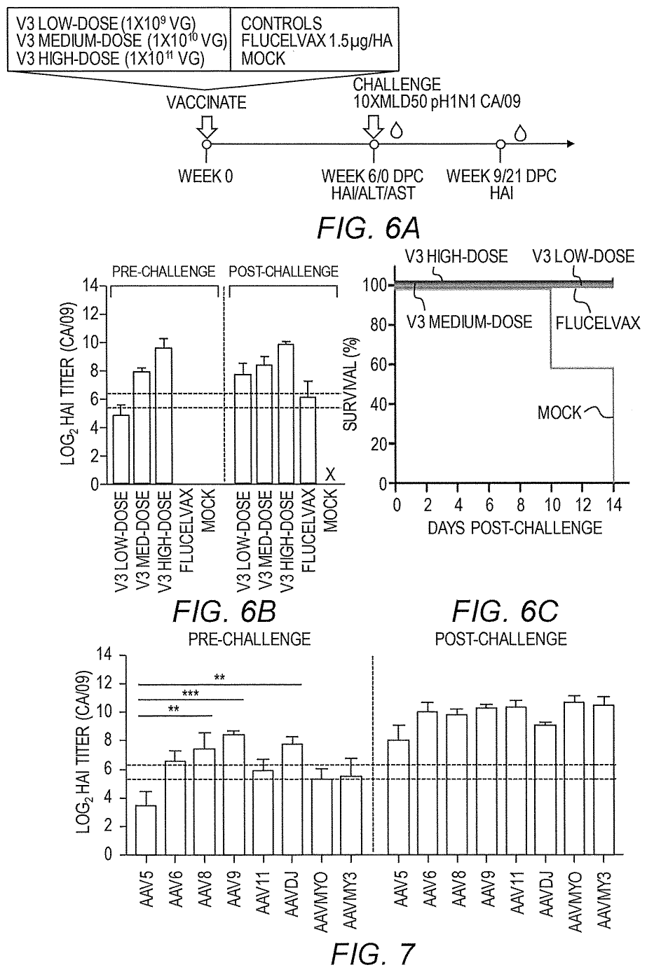

Sex-specific differences in AAV transgene expression (female mice have lower expression) have been previously shown. In addition, it is known that female mice are more susceptible to severe influenza disease. In this study, it was observed that the COBRA HA antigen vaccine (V3 vaccine, FIG. 4A) was superior to FLUCELVAX® seasonal influenza vaccine in female mice at all doses, the lowest tested being the 1×10⁹ vg dose (FIGS. 6A-6C).

### Example 5: Vaccines with Different Capsids

Vaccines designed with different capsids were prepared. All vaccines were administered intramuscularly at 1×10⁹ vg. Each protected the mice from a lethal viral challenge, resulting in 100% survival. However, moderate weight loss was observed with some capsids. These differences trend with pre-challenge HAI titers (FIG. 7), a good proxy for the immune response against the HA transgene. These data indicate that in cases where a subject may have already been exposed to a given serotype (and have neutralizing antibodies), an evolutionarily divergent or synthetically engineered capsid may be used to evade the immune response generated against that capsid to which the subject was previously exposed.

### Example 6: Protection in a Ferret Model

In this study, efficacy in a ferret model was demonstrated. Notably, the ferret model is the gold standard for influenza studies. The ferrets had previously been challenged with different influenza strains to generate pre-existing influenza immunity. Overcoming this is challenging for many vaccine platforms. Initial exposures to influenza are described as “original antigenic sin,” which biases future immune responses from vaccination toward epitopes from older strains of influenza. In this study, it was demonstrated that AAV vaccination of ferrets with ssAAV9-COBRA HA vaccine boosts their antibody responses toward a broad set of H1N1 strains including California/09, Michigan/15, Idaho/18 and Hawaii/19 compared to vaccination with ssAAV2/9-GFP.

### Example 7: CpG Titration

Unmodified ssAAV2/9-COBRA HA (not CpG enriched) completely protects mice from mortality at 1×10¹⁰ and 1×10⁹ vg, but morbidity (as measured by weight loss and clinical scores) and mortality (as measured by survival) are observed at lower doses (1×10⁶, 1×10⁷, and 1×10⁸). There is a protective effect at 1×10⁸ and 1×10⁷ vg, but the mice that cannot recover from the viral challenge must be euthanized at the end of the study due to sustained weight loss and clinical scores.

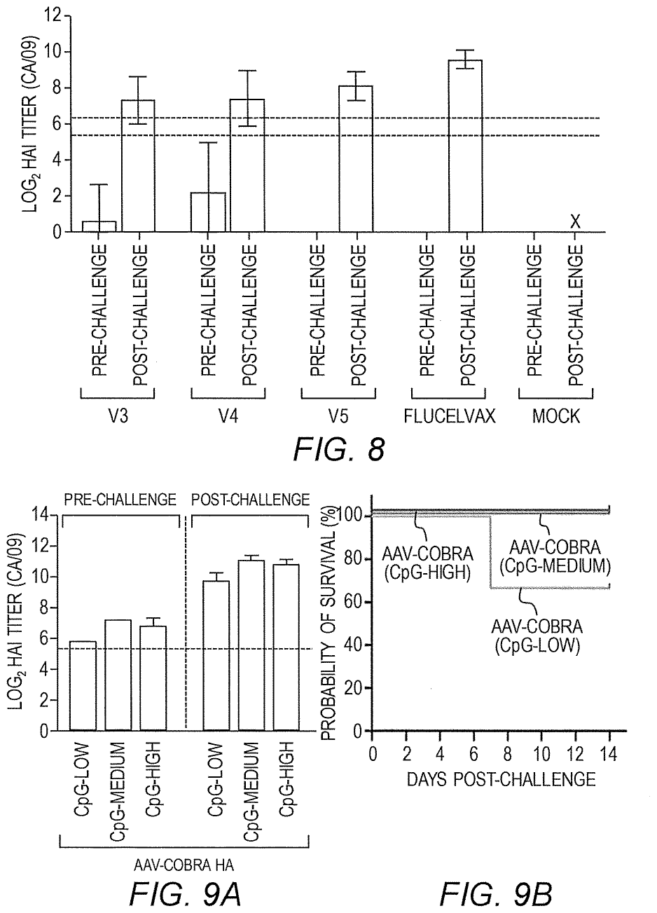

Based on the results with unmodified vector, it was determined whether CpG enrichment within the COBRA HA antigen could improve vaccine performance and allow for lower dosing. For this analysis, mice were vaccinated with 5×10⁸ vg (as determined by the dose de-escalation study as the lower limit of protection) of CpG-Low (V3: ssAAV2/9-COBRA HA vector, 162 total CpG), CpG-Medium (V4: ssAAV2/9-COBRA HA vector, 242 total CpG), and CpG-High (V5: ssAAV2/9-COBRA HA vector, 345 total CpG) vectors. With the increased CpG-content, strong efficacy at this lower dose was observed (FIG. 8) based on minimal weight loss and complete survival following a lethal viral challenge. Therefore, complete protection against mortality may be attained at doses below 5×10⁸ vg using these enriched vectors.

### Example 8: T-Cell Responses

Mice were vaccinated with 5×10⁸ vg dose of the CpG-Low (V3: ssAAV2/9-COBRA HA vector, 162 total CpG), CpG-Medium (V4: ssAAV2/9-COBRA HA vector, 242 total CpG), and CpG-High (V5: ssAAV2/9-COBRA HA vector, 345 total CpG) versions of the AAV COBRA HA vector. Following vaccination, T-cell responses were assessed in the lungs and spleens. The results of this analysis indicated that CpG enrichment AAV vector increased T-cell responses (TNFα, IFNγ, and/or IL-2 interferon-producing CD4+ and CD8+ T-cells) specifically to the hemagglutinin head. While not statistically significant, the CpG-High group always trended toward higher frequencies in both CD4+ and CD8+ T-cell responses. Therefore, CpG enrichment of antigen coding sequence may increase the frequency of multiple influenza-reactive T cell populations in both lungs and spleens.

### Example 9: Intranasal Administration

As demonstrated in, e.g., Examples 1-2, HA antigen vaccines of this invention were successfully administered by the intramuscular administration route. To expand the use of the vaccine, it was determined whether an immune response could be elicited by intranasal administration. Mice were vaccinated with 2.5×10¹⁰ vg intranasally, and it was found that the mice mounted seroprotective antibody titers (FIG. 9A). In addition, increased CpG content was observed to offer improved survival rates via this route (FIG. 9B).

### Example 10: Broad Spectrum Antibody Responses

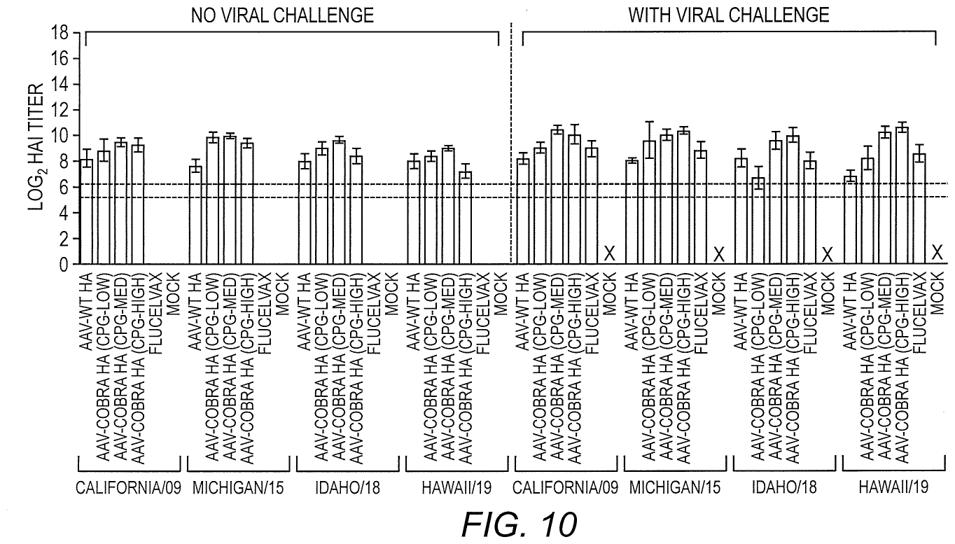

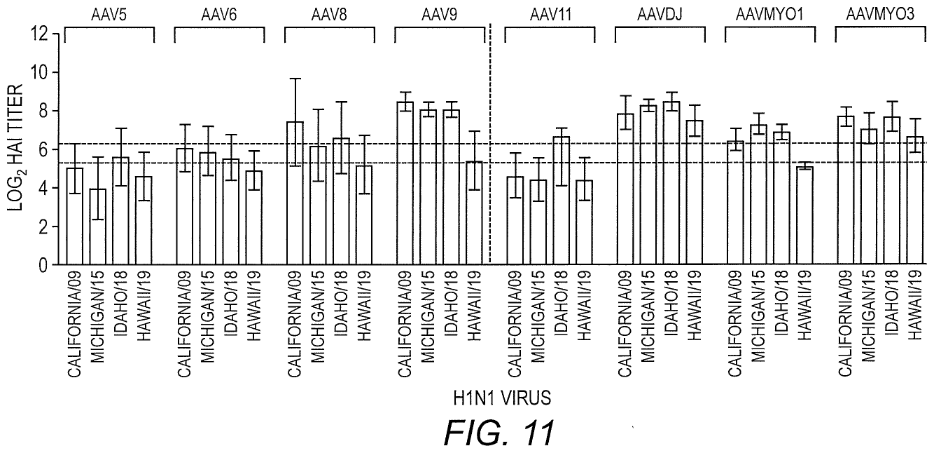

COBRA HA delivered via the AAV vector platform provided a better breadth of antibody responses against other H1N1 strains compared to the antibody response against wild-type HA (FIG. 10). In addition, there was a robust breadth of antibody responses regardless of capsid, although some capsids resulted in stronger responses than others (FIG. 11).

### Example 11: Vaccines Including Multiple Antigens

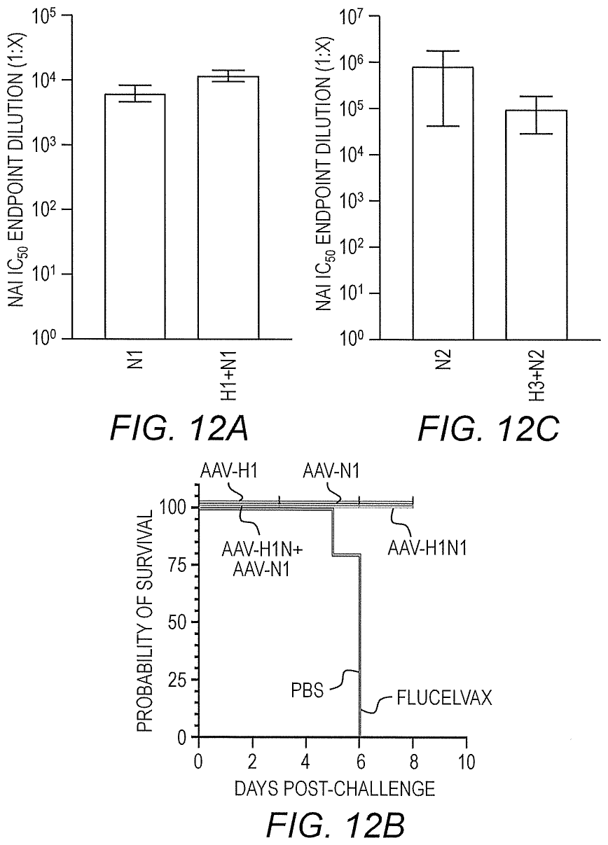

In addition to vectors expressing HA, it was determined whether the AAV vector platform could be used to express other antigens. In particular, it was determined whether NA antibodies could be elicited when the AAV vector expresses N1. C57BL/6J mice were injected intramuscularly with 1×10¹⁰ vg of the following vector combinations: ssAAV2/9 COBRA H1; ssAAV2/9 COBRA N1; ssAAV2/9 COBRA H1+ssAAV2/9 COBRA N1 (5×10⁹ vg each); or ssAAV2/9 COBRA H1N1. Mice were challenged 10.5 weeks post-vaccination intranasally with 10XMLD₅₀ CA/09 (pH1N1) virus. This analysis indicated that pooling two AAV vectors, one encoding the H1 antigen and another encoding N1, is more efficacious than H1N1 on the same vector. In particular, all AAV vector combinations protect against severe weight loss and lethality (FIG. 12A), with the best combination being the H1+N1 combination. Notably, NAI activity was observed in pre-challenge sera (FIG. As such, pooling antigens/vectors does not diminish the response against each individual antigen.

In a similar set of experiments, C57BL/6J mice were injected intramuscularly with 1×10¹⁰ vg of the following vector combinations: ssAAV2/9 COBRA H3; ssAAV2/9 COBRA N2; SSAAV2/9 COBRA H3+ssAAV2/9 COBRA N2 (5×10⁹ vg each); or SSAAV2/9 COBRA H3N2. Mice were challenged 10.5 weeks post-vaccination intranasally with a mouse-adapted Switzerland/13 (H3N2) virus known to cause morbidity but not lethality. This analysis indicated that all AAV vector combinations protected against weight loss and provided complete protection against mortality. Notably, NAI activity was observed in pre-challenge sera (FIG. 12C). In addition to exhibiting efficacy when delivered via separate vectors, COBRA H3 and N2 were also efficacious when provided on the same vector.

What is claimed is:

1. A recombinant, non-replicating Adeno-Associated Virus (AAV) vector comprising a promoter operably linked to nucleic acids encoding one or more immunogenic peptides or proteins, wherein said nucleic acids comprise a plurality of immunostimulatory CpG motifs therein.

2. The vector of claim 1, wherein the promoter further comprises a plurality of immunostimulatory CpG motifs.

3. The vector of claim 1, wherein the AAV is a single-stranded AAV (ssAAV) vector or a self-complementary AAV vector (scAAV).

4. The vector of claim 1, wherein the promoter is a tissue-specific promoter.

5. The vector of claim 4, wherein the tissue-specific promoter is a lung-specific promoter or muscle-specific promoter.

6. The vector of claim 1, wherein the immunogenic peptides are computationally optimized broadly reactive antigens (COBRAs).

7. The vector of claim 1, wherein the immunogenic peptides or proteins are Influenza virus antigens.

8. The vector of claim 7, wherein the Influenza virus antigens comprise hemagglutinin, neuraminidase, nucleoprotein, matrix antigen, or a combination thereof.

9. The vector of claim 7, wherein the Influenza virus antigens are monovalent or multivalent.

10. The vector of claim 1, wherein the nucleic acids encoding the immunogenic peptides or proteins are located:
(i) downstream of promoter P5 of the AAV vector; and/or
(ii) upstream of promoter P5 or 5′ inverted terminal repeat sequences of the AAV vector.

11. An immunogenic composition comprising the vector of claim 1 in admixture with an acceptable carrier, wherein said immunogenic composition does not include an exogenous adjuvant.

12. The immunogenic composition of claim 11, wherein the immunogenic composition is formulated for intramuscular, intradermal, or intranasal administration.

13. A method of eliciting an immune response in a subject comprising administering an immunogenic composition of claim 11 to the subject, thereby eliciting an immune response in the subject.

14. The method of claim 13, wherein the immune response is a humoral response.

15. The method of claim 14, wherein the humoral response comprises neutralizing antibodies.
